SEQUENCE LISTING (2 sequences)

SEQ ID NO: 1 - protein, 23 aa, synthetic construct
KKQQQQQQQQQQQQQQQQQQQQK

SEQ ID NO: 2 - protein, 40 aa, synthetic construct
DAEFRHDSGYEVHHQKLVFFAEDVGSNKGAIIGLMVGGVV

(19) United States
(12) Patent Application Publication
(10) Pub. No.: US 2023/0174561 A1
(43) Pub. Date: Jun. 8, 2023

(54) IONIC COMPOUND AND COMPOSITION THEREOF FOR HYDROPHOBIC PROTEIN EXTRACTION; AND ANTI-NONSPECIFIC PROTEIN ADSORPTION ZWITTERIONIC COMPOUND AND ELEMENT WITH ANTI-NONSPECIFIC PROTEIN ADSORPTION LAYER COMPRISING THE SAME

(71) Applicant: YANQIN HOLDING COMPANY, Tainan City (TW)

(72) Inventor: Hui-Yin HUANG, Tainan City (TW)

(21) Appl. No.: 18/073,703

(22) Filed: Dec. 2, 2022

Related U.S. Application Data
(60) Provisional application No. 63/285,260, filed on Dec. 2, 2021.

(51) Int. Cl. C07F 7/18; C07D 233/61; B01J 20/22; C07K 1/14; C07K 14/415; B01J 20/28

(52) U.S. Cl. CPC C07F 7/1804; C07D 233/61; B01J 20/22; C07K 1/145; C07K 14/415; B01J 20/28035; B01J 2220/46

(57) ABSTRACT

Provided is an ionic compound, which can be formulated into an ionic solution for extracting hydrophobic proteins. The ionic solution is free of alcohol and thus advantageous for later examinations. Also provided is an anti-nonspecific protein adsorption zwitterionic compound, which can be coated on a substrate to form an anti-nonspecific protein adsorption element.

### CROSS-REFERENCE TO RELATED APPLICATION

Pursuant to 35 U.S.C. § 119(e), this application claims the benefit of the priority to U.S. Provisional Patent Application No. 63/285,260, filed on Dec. 2, 2021. The content of the prior application is incorporated herein by its entirety.

### REFERENCE TO AN ELECTRONIC SEQUENCE LISTING

The content of the electronic sequence listing (CFP033411.101.xml; Size: (2,840 bytes; and Date of Creation: Nov. 23, 2022) is herein incorporated by reference in its entirety.

### BACKGROUND OF THE INVENTION

### 1. Field of the Invention

The present invention relates to an ionic compound for extracting hydrophobic proteins and an ionic solution formulated by the ionic compound. In addition, the present invention relates to an anti-nonspecific protein adsorption zwitterionic compound and an element comprising a coating layer formed by the zwitterionic compound.

### 2. Description of the Prior Arts

Proteins are complicated large molecules, and protein examination in the biochemical field is still challenging in the current years. To examine hydrophilic proteins, an alcohol solution, such as 75 wt % ethanol solution, is used for protein extraction. However, the presence of alcohol is disadvantageous for later examination such as ELISA in which antigen-antibody interaction is involved. Therefore, the development of new extraction medium is still needed. In addition, the nonspecific protein adsorption is also a serious issue for protein examination and need to be improved.

### SUMMARY OF THE INVENTION

To overcome the shortcomings, one objective of the present invention is to provide an ionic compound, which can be formulated into an ionic solution for extracting hydrophobic proteins, especially gliadin, mutant Huntingtin protein (mHtt), or beta-amyloid protein.

Another objective of the present invention is to provide an anti-nonspecific protein adsorption zwitterionic compound, which can be coated on an element to form an anti-nonspecific protein adsorption layer, and the anti-nonspecific protein adsorption layer has a contact angle of 10° or less.

To achieve the above objectives, the present invention provides an ionic compound represented by the following Formula I:

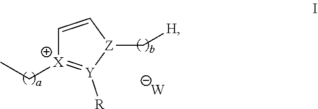

wherein,
X is N;
Y is C or N;
Z is N;
R is H, —CH₃, —C₂H₅, —C₃H₇ or —C₄H₉;
a is an integer of 1 to 40;
b is an integer of 0 to 40; and
W⁻ is selected from the group consisting of F⁻, Cl⁻, OMs⁻, NTf₂⁻, HSO₃⁻, HSO₄⁻, NO₃⁻, NO₂⁻ and H₂PO₄⁻.

In some embodiments, Y is C. In some embodiments, X is N. In some embodiments, Z is N. In some embodiments, Y is C; X is N; and Z is N.

In some embodiments, a is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In some embodiments, a is an integer of 3 to 15, or 5 to 12, or 5 to 7.

In some embodiments, b is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. In some embodiments, b is an integer of 0 to 5, or 1 to 3.

In some embodiments, W⁻ is OMs⁻, HSO₄⁻ or NO₃⁻.

The present invention also provides an ionic solution for extracting a hydrophobic protein, comprising the above-mentioned ionic compound and a solvent.

In some embodiments, the solvent is water or a water-based buffer. In some embodiments, the water-based buffer is phosphate buffered saline (PBS).

In some embodiments, the ionic compound is in an amount of 0.1% by weight (wt %) to 10 wt %, or 0.5 wt % to 2 wt %, or 0.5 wt % to 1 wt %.

In some embodiments, the hydrophobic protein is gliadin, mutant Huntingtin protein (mHtt), or beta-amyloid protein ((αβ40).

Yet, the present invention provides an anti-nonspecific protein adsorption zwitterionic compound represented by the following Formula II:

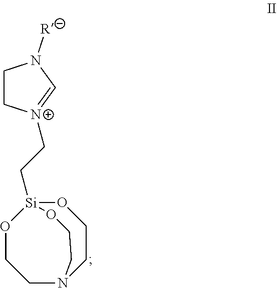

wherein,
R′ is

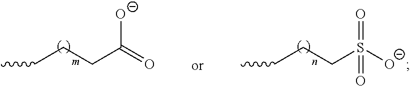

m is an integer of 0 to 7; and
n is an integer of 1 to 7.

In some embodiments, m is 1, 2, 3, 4, 5, 6 or 7. In some embodiments, R′ is

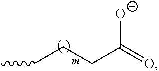

and m is an integer of 0 to 3, or 1 to 2.

In some embodiments, R′ is

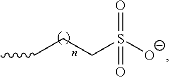

and n is an integer of 1 to 3,or 1 to 2.

The present invention also provides an anti-nonspecific protein adsorption element comprising a substrate and an anti-nonspecific protein adsorption layer, wherein the substrate is coated with the above-mentioned zwitterionic compound to form the anti-nonspecific protein adsorption layer.

In some embodiments, the substrate is made of plastic, metal or metal oxide. In some embodiments, the plastic is polypropylene (PP), polyvinylchloride (PVC) or polystyrene. In some embodiments, the metal is gold. In some embodiments, the metal oxide is iron(III) oxide (Fe₂O₃), iron(II,III) oxide (Fe₃O₄) or aluminum oxide (Al₂O₃).

In some embodiments, the anti-nonspecific protein adsorption layer has a coverage rate of 80% to 100% on the substrate. In some embodiments, the anti-nonspecific protein adsorption layer has a coverage rate of 99% to 100% on the substrate. In some embodiments, the anti-nonspecific protein adsorption layer has a coverage rate of 99.5% to 100% on the substrate.

In some embodiments, the anti-nonspecific protein adsorption layer has a contact angle of or 10° or less, or 5° to 10°, which is examined by a contact angle meter. In some embodiments, the contact angle is examined on the surface of the anti-nonspecific protein adsorption layer, which is opposite to the bottom contacted with the substrate.

### BRIEF DESCRIPTION OF THE DRAWINGS

FIG. 1 is a bar chart showing the concentration and solubility of gliadin in the [DMePIm][OMs] ionic solutions at different concentrations.

FIG. 2 is a line chart showing the concentration and solubility of gliadin in 1 wt % [DMePIm][OMs] ionic solution over time.

FIG. 3 is a dot chart showing the concentration and solubility of gliadin in ionic solutions comprising ionic compound with different cations.

FIG. 4 is a dot chart showing the concentration and solubility of gliadin in ionic solutions comprising ionic compound with different anions.

FIG. 5 is a dot chart showing gliadin concentration can be determined by the method designed in Test Example 5.

FIG. 6 is a line chart showing the structure of gliadin is the same before and after the extraction with 1 wt % [DMePIm][OMs] ionic solution.

FIG. 7 is a bar chart showing [DMePIm][OMs] has good biocompatibility.

FIG. 8 is a dot chart showing the relationship between increase of the coverage rate of Z1 Compound and protein adsorption rate.

FIG. 9 is a dot chart showing the relationship between the contact angle of surface of the coating layer form by Z1 Compound and the coverage rate.

FIG. 10 is a line chart showing the coating layer formed by Z1 Compound is not deteriorated by strong alkali treatment.

FIG. 11 is a line chart showing the coating layers formed by Z1 Compound and the Q-amine compound had a hydrophilic characteristic when the contact angle is tested with PBS solutions with pH ranging from 2 to 12.

FIG. 12 is a line chart showing the hydrophilic characteristic of the coating layers formed by the Q-amine compound is damaged by strong alkali treatment and the damage is more obvious when the contact angle is tested with a PBS solution having a higher pH value, but such damage is not observed at the coating layer formed by Z1 Compound.

FIG. 13 is a line chart showing the coating layer formed by the Z1 compound could be applied to biochemical analysis.

### DETAILED DESCRIPTION OF THE PREFERRED EMBODIMENTS

Other objectives, advantages and novel features of the invention will become more apparent from the following detailed description when taken in conjunction with the accompanying drawings.

### Synthesis of Ionic Compounds of the Present Invention

### Synthesis Example 1-1: [PIm][OMs]

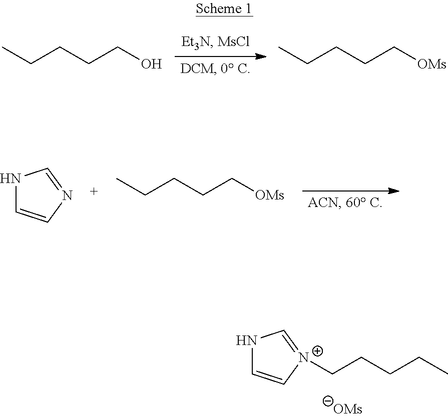

As shown in Scheme 1, 1 equivalence (eq) of 1-pentanol and 1.2 eq of triethylamine were introduced into a reaction bulb, and then 20 milliliters (mL) of dichloromethane (DCM) was added, and stirred in an ice bath for 5 minutes (min). 1.1 eq of methanesulfonyl chloride (MsCl) was slowly dropped into the reaction bulb to give a mixture. After that, the ice bath was removed, and the mixture was reacted at room temperature for 20 min to give a first reaction mixture.

The first reaction mixture was extracted by 10% (w/v) citric acid aqueous solution for three times (water phase), and then extracted by 10% sodium hydrogen carbonate (NaHCO₃) aqueous solution for three times to give an extract. After extraction, the solvent comprised in the extract was removed by concentration under reduced pressure. 0.9 eq of imidazole and 50 mL of acetonitrile (ACN) were added and heated to react at 60° C. for 12 hours (h) to give a second reaction mixture. After that, the solvent comprised in the second reaction mixture was removed by concentration under reduced pressure to give a first crude product. The first crude product was extracted by hexane, and dried by concentration under reduced pressure to give [3-pentyl imidazolium][methanesulfonyl] ([PIm][OMs]).

NMR (200 MHz, D₂O): 0.85 (3H, t), 1.24-1.26 (2H, m), 1.28-1.31 (2H, m), 2.01-2.04 (2H, m), 4.98-5.02 (2H, t), 7.44 (1H, d), 7.56 (1H, d), 8.86 (1H, s)

### Synthesis Example 1-2: [HpIm][OMs]

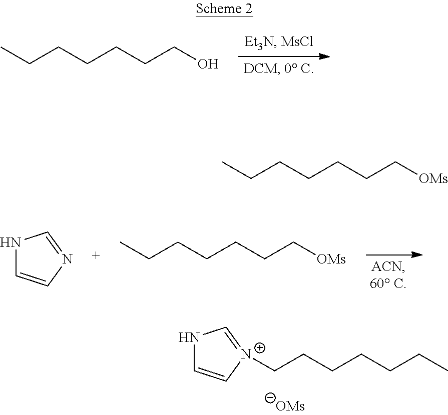

[3-heptyl imidazolium][methanesulfonyl] ([HpIm][OMs]) was prepared by the same method as described in Synthesis Example 1-1, except the 1-pentanol was replaced by 1-heptanol.

NMR (200 MHz, D₂O): 0.84-0.88 (3H, t), 1.26-1.35 (8H, m), 1.89-2.03 (2H, m), 5.01-5.04 (2H, t), 7.45-7.48 (1H, d), 7.57-7.59 (1H, d), 8.91 (1H, s)

In addition, similar compounds with a group having a different carbon number on 3-position of the imidazolium (value “a” of Formula I) could be prepared in accordance with Synthesis Examples 1-1 and 1-2 except using an alcohol having a different carbon length.

### Synthesis Example 1-3: [MePIm][OMs]

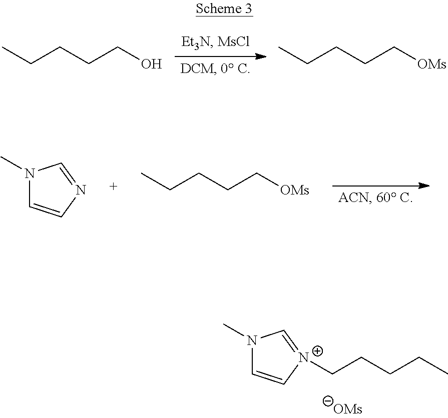

[1-methyl, 3-pentyl imidazolium][methanesulfonyl] ([MePIm][OMs]) was prepared by the same method as described in Synthesis Example 1-1, except the imidazole was replaced by 1-methyl imidazole.

NMR (200 MHz, D₂O): 0.82 (3H, t), 1.22-1.24 (2H, m), 1.30-1.31 (2H, m), 1.98-2.01 (2H, m), 3.72 (3H, s), 5.02-5.08 (2H, t), 7.45 (1H, d), 7.58 (1H, d), 8.85 (1H, s)

In addition, similar compounds with a group having a different carbon number on 3-position of the 1-dimethyl imidazolium (value “a” of Formula I) can be prepared in accordance with Synthesis Example 1-3 except using an alcohol having a different carbon length.

### Synthesis Example 1-4: [DMePIm][OMs]

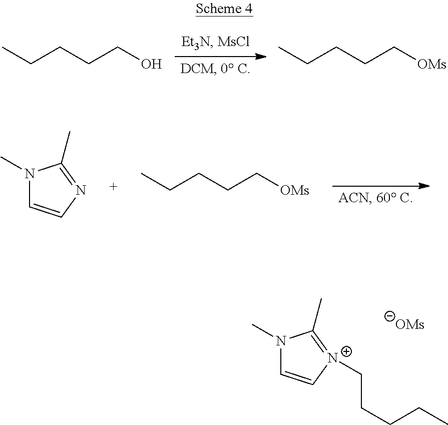

[1,2-dimethyl, 3-pentyl imidazolium][methanesulfonyl] ([DMePIm][OMs]) was prepared by the same method as described in Synthesis Example 1-1, except the imidazole was replaced by 1,2-dimethyl imidazole.

NMR (200 MHz, D₂O): 0.86-0.88 (3H, t), 1.86-1.91 (4H, m), 3.85-3.87 (2H, t), 3.91 (3H, s), 4.12 (3H, s), 7.84-7.85 (1H, d), 7.87-7.88 (1H, s).

In addition, similar compounds with a group having a different carbon number on 3-position of the 1,2-dimethyl imidazolium (value “a” of Formula I) can be prepared in accordance with Synthesis Example 1-4 except using an alcohol having a different carbon length. In the present invention, 1 eq of 1,2-dimethyl imidazole and 1 eq of methanesulfinic acid were mixed in water to react in an ice bath for 5 min to give a third crude product, and the third crude product was lyophilized to obtain [1,2-dimethyl imidazolium] [methanesulfonyl] ([DMeIm][OMs]). In addition, methanol (C1), propanol (C3), heptanol (C7), nonanol (C9), or dodecanol (C12) were used to synthesize the following compounds:
1. [1,2-dimethyl imidazolium][methanesulfonyl] ([DMeIm][OMs])

NMR (DCCl₃): 2.48 (3H, s), 3.52 (3H, s), 6.82-6.84 (1H, d), 6.94-6.96 (1H, s)
2. [1,2,3-trimethyl imidazolium][methanesulfonyl] ([TMeIm][OMs])

NMR (DCCl₃): 2.8 (3H, s) 4.0 (6H, s), 7.4-7.6 (1H, d), 7.8-7.9 (1H, d)
3. [1,2-dimethyl, 3-propyl imidazolium][methanesulfonyl] ([DMePrIm][OMs])

NMR (DCCl₃): 0.86-0.88 (3H, t), 1.89-1.91 (2H, t), 3.84-3.85 (2H, t), 3.91 (3H, s), 4.11 (3H, s), 7.64-7.65 (1H, d), 7.83-7.84 (1H, s).
4. [1,2-dimethyl, 3-heptyl imidazolium][methanesulfonyl] ([DMeHpIm][OMs])
NMR (D₂O): 0.86-0.88 (3H, t), 1.22-1.29 (10H, m), 3.48-3.50 (2H, t), 4.01 (3H, s), 4.28 (3H, s), 7.85-7.87 (2H, d)
5. [1,2-dimethyl, 3-nonyl imidazolium][methanesulfonyl] ([DMeNnIm][OMs])

NMR (D₂O): 0.84-0.86 (3H, t), 1.20-1.28 (12H, m), 1.84-1.87 (2H, m), 2.95 (3H, s), 4.02-4.03 (2H, t), 4.36 (3H, s), 7.88-7.90 (2H, d)
6. [1,2-dimethyl, 3-dodecyl imidazolium][methanesulfonyl] ([DMeDdIm][OMs])
NMR (DCCl₃): 0.86-0.88 (3H, t), 1.80-1.91 (18H, m), 3.84-3.85 (2H, t), 3.91 (3H, s), 4.11 (3H, s), 7.83-7.84 (2H, d).

### Synthesis Example 1-5: [DMePIm][Cl]

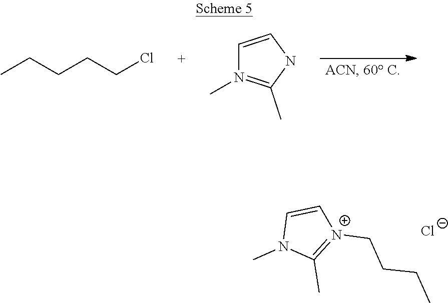

1 eq of 1-chloropentane was added to 0.9 eq of 1,2-dimethyl imidazole and 50 mL of acetonitrile (ACN) in a reaction bulb, and heated to react at 60° C. for 12 h to give a third reaction mixture. After that, the solvent comprised in the third reaction mixture was removed by concentration under reduced pressure to give a second crude product. The second crude product was extracted by hexane, and dried by concentration under reduced pressure to give [1,2-dimethyl, 3-pentyl imidazolium][chloride ion] ([DMePIm][Cl]).

NMR (DCCl₃): 0.87-0.89 (3H, t), 1.85-1.91 (4H, m), 3.83-3.86 (2H, t), 3.95 (3H, s), 4.13 (3H, s), 7.85-7.86 (1H, d), 7.87-7.88 (1H, s).

Similar compounds with another halogen anion can be prepared in accordance with Synthesis Example 1-5 except using another halogenated pentane. In the present invention, 1-fluoropentane was used to give [1,2-dimethyl, 3-pentyl imidazolium][fluoride ion] ([DMePIm][F]).

In addition, the anion can be further changed by addition of a silver salt after the formation of [DMePIm][Cl] or [DMePIm][F]. In the present invention, silver nitrate (AgNO₃), silver dihydrogen phosphate (AgH₂PO₄) and silver hydrogen sulfate (AgHSO₄) were respectively added into [DMePIm][Cl] aqueous solution obtained from Synthesis Examples 1-5 to give silver chloride and [DMePIm][NO₃], [DMePIm][H₂PO₄] or [DMePIm][HSO₄]. Therefore, the following compounds were also synthesized:
1. [1,2-dimethyl, 3-pentyl imidazolium][fluoride ion] ([DMePIm][F])

NMR (DCCl₃): 0.87-0.89 (3H, t), 1.85-1.91 (4H, m), 3.83-3.86 (2H, t), 3.95 (3H, s), 4.25 (3H, s), 7.85-7.86 (1H, d), 7.87-7.88 (1H, s).
2. [1,2-dimethyl, 3-pentyl imidazolium][nitrate ion] ([DMePIm][NO3])
NMR (D₂O): 0.86-0.88 (3H, t), 1.23-1.26 (8H, m), 1,27-1.30 (2H, t), 4.01 (3H, s), 4.28 (3H, s), 7.86-7.87 (2H, d)
3. [1,2-dimethyl, 3-pentyl imidazolium][dihydrogen phosphate ion] ([DMePIm][H₂PO₄])
NMR (D₂O): 0.86-0.88 (3H, t), 1.22-1.27 (8H, m), 1,28-1.31 (2H, t), 4.03 (3H, s), 4.26 (3H, s), 7.85-7.87 (2H, d)
4. [1,2-dimethyl, 3-pentyl imidazolium][hydrogen sulfate ion] ([DMePIm][H₂PO₄])
NMR (D₂O): 0.86-0.89 (3H, t), 1.22-1.26 (8H, m), 1,28-1.32 (2H, t), 4.02 (3H, s), 4.26 (3H, s), 7.85-7.87 (2H, d)

The following compounds were synthesized in accordance with Synthesis Examples 1-1 to 1-5:

TABLE 1

| | Positions of the | | | |
|---|---|---|---|---|
| | imidazolium (cation) | | | |
| Compound | 1 | 2 | 3 | Anion |
| [PIm][OMs] | H | H | pentyl | methanesulfonyl |
| | | | (C5) | |
| [HIm][OMs] | H | H | heptyl | methanesulfonyl |
| | | | (C7) | |
| [MePIm][OMs] | methyl | H | pentyl | methanesulfonyl |
| | (C1) | | (C5) | |
| [DMeIm][OMs] | methyl | methyl | H | methanesulfonyl |
| | (C1) | (C1) | | |
| [TMeIm][OMs] | methyl | methyl | methyl | methanesulfonyl |
| | (C1) | (C1) | (C1) | |
| [DMePrIm][OMs] | methyl | methyl | propyl | methanesulfonyl |
| | (C1) | (C1) | (C3) | |
| [DMePIm][OMs] | methyl | methyl | pentyl | methanesulfonyl |
| | (C1) | (C1) | (C5) | |
| [DMeHpIm][OMs] | methyl | methyl | heptyl | methanesulfonyl |
| | (C1) | (C1) | (C7) | |
| [DMeNnIm][OMs] | methyl | methyl | nonyl | methanesulfonyl |
| | (C1) | (C1) | (C9) | |
| [DMeDdIm][OMs] | methyl | methyl | dodecyl | methanesulfonyl |
| | (C1) | (C1) | (C12) | |
| [DMePIm][C1] | methyl | methyl | pentyl | chloride ion |
| | (C1) | (C1) | (C5) | |
| [DMePIm][F] | methyl | methyl | pentyl | fluoride ion |
| | (C1) | (C1) | (C5) | |
| [DMePIm][NO₃] | methyl | methyl | pentyl | nitrate ion |
| | (C1) | (C1) | (C5) | |
| [DMePIm][H₂PO₄] | methyl | methyl | pentyl | dihydrogen |
| | (C1) | (C1) | (C5) | phosphate ion |
| [DMePIm][HSO₄] | methyl | methyl | pentyl | hydrogen |
| | (C1) | (C1) | (C5) | sulfate ion |

### Synthesis of Zwitterionic Compounds of the Present Invention

### Synthesis Example 2-1: Z1 Compound

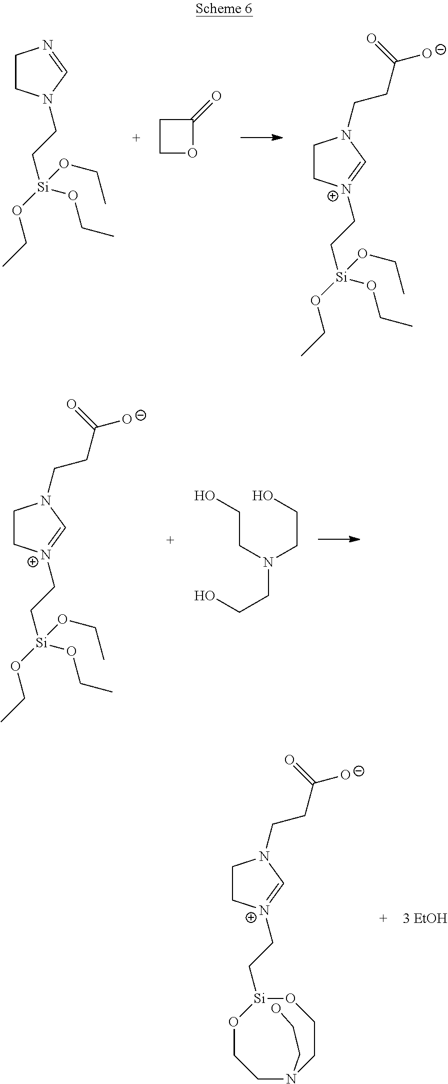

1 eq of triethoxy-3-(2-imidazolin-1-yl)-propylsilane, 1.1 eq of beta-propiolactone and 20 mL of acetonitrile were introduced into a reaction bulb, heated under reflux for deoxgenation for 12 h to give a fourth reaction mixture. After reaction, the solvent comprised in the fourth reaction mixture was removed by concentration under reduced pressure to give a crude intermediate product. The crude intermediate product was washed by ice-cold ether for several times, and then the solvent was removed again by concentration under reduced pressure to give an intermediate product.

After that, 0.9 eq of triethylamine and 50 mL of toluene were added, and refluxed for 24 h in a Dean-Stark apparatus to dewater. After that, the Dean-Stark apparatus was removed and acetone was added to wash out unreacted components and obtain Z1 compound.

NMR (d-DMSO): 0.56 (2H, t), 1.66 (2H, m), 1.92 (2H, t), 2.42 (2H, t), 2.62 (6H, t), 3.16 (2H, t), 3.88 (6H, t), 4.08 (2H, t), 7.72 (1H, s)

### Synthesis Example 2-2: Q-Amine Compound

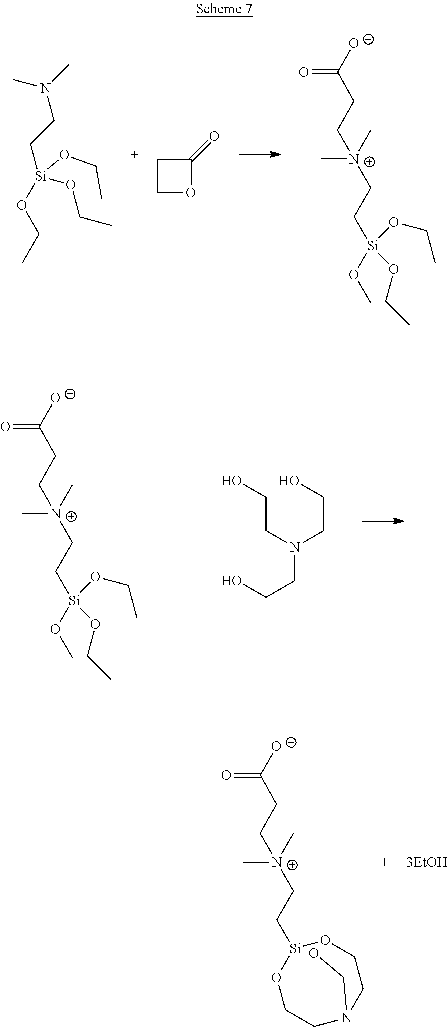

The Q-amine compound was prepared by the same method as described in Synthesis Example 2-1, except the triethoxy-3-(2-imidazolin-1-yl)-propylsilane was replaced by N,N-dimethyl-2-(triethoxysilyl)ethan-1-amine (TCI, CAS: 65411-99-6). The Q-amine compound is a quaternary ammonium compound with similar structure with the Z1 compound of the present invention. Quaternary ammonium compounds are traditionally used for anti-nonspecific protein adsorption. This Q-amine compound was designed and synthesized for comparison of anti-nonspecific protein adsorption effect in the present invention.

NMR (d-DMSO): 0.56-1.57 (2H, t), 2.57-2.60 6H. t), 2.83-2.85 (2H, t), 3.21-3.23 (2H, m), 3.29 (6H, s), 3.56-3.57 (2H, t), 3.86-3.89 (6H, t)

### Tests for Ionic Compounds of the Present Invention

Gliadin is a hydrophobic protein present in gluten of wheat. Unless specifically stated, gliadin (G3375, Sigma-Aldrich; CAS No. 9007-90-3) or was used in the following tests.

### Test Example 1: Solubility of Gliadin in [DMePIm][OMs] Ionic Solution

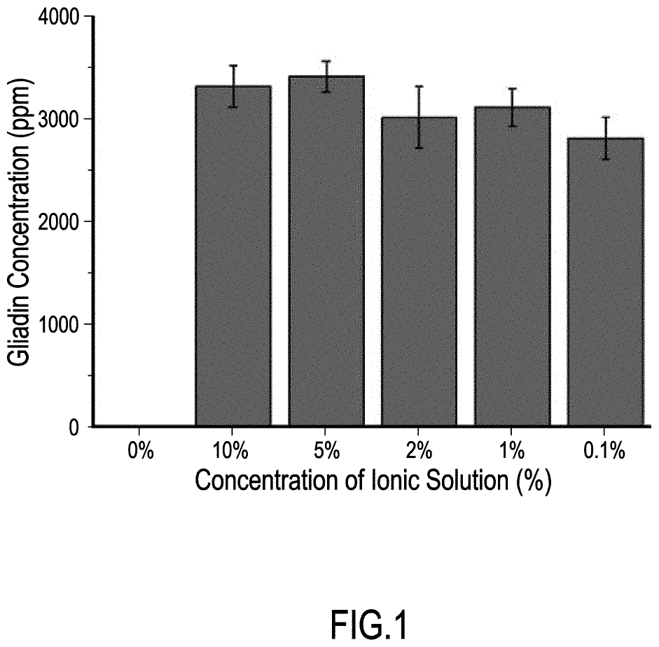

First of all, ionic solutions of [DMePIm][OMs] with a concentration of 0.1 wt %, 1 wt %, 2 wt %, 5 wt % and 10 wt % in water were prepared. 3 grams (g) of gliadin (excess amount) was added into 10 mL of each [DMePIm][OMs] ionic solution, mixed homogenously and stood still for 5 min to give sample solutions for Test Example 1. All sample solutions were centrifuged at 8500 rpm for 3 min and filtered with a filter with 0.22 μm pore size. Water (0 wt %) was used as the control group. After that, Wheat/Gluten (Gliadin) ELISA Kit (Crystal chem, AOAC No. 011804) was used to determine the concentration and solubility of gliadin. Since the examination of the Wheat/Gluten (Gliadin) ELISA Kit is about 100 ppm, the gliadin concentration was determined by diluted sample solutions with at least 50-fold dilution by the corresponding ionic solution, in order to calculate the gliadin concentration of the sample solutions. As shown in FIG. 1, gliadin had solubility values of about 3000 ppm in the [DMePIm][OMs] ionic solutions at all concentrations.

### Test Example 2: Solubility of Gliadin in 1 wt % [DMePIm][OMs] Ionic Solution over Time

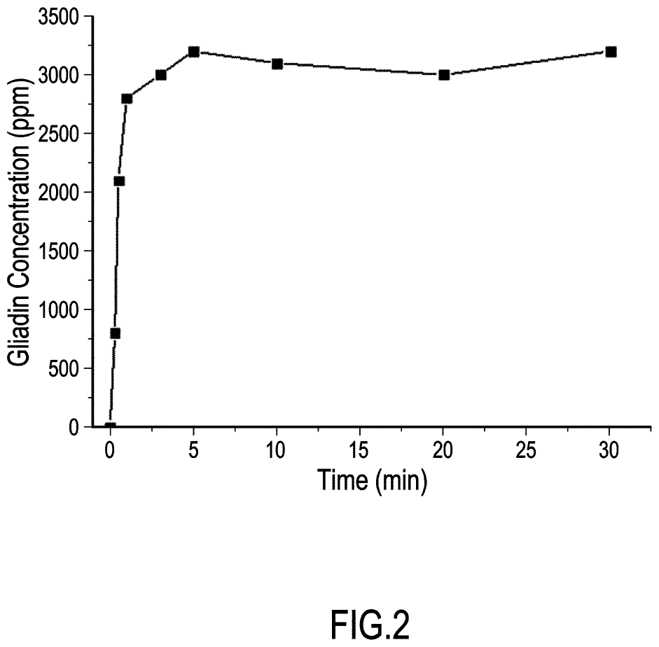

Similar with Test Example 1, except that 3 g of gliadin was added into 10 mL of 1 wt % [DMePIm][OMs] ionic solution, and the concentration and solubility was tested after 1 min, 2 min, 3 min, 4 min, 5 min, 10 min, 20 min and 30 min. As shown in FIG. 2, the concentration of gliadin reached its maximum solubility shortly.

### Test Example 3: Solubility of Gliadin in 1 wt % Ionic Solutions of Ionic Compounds with Different Cations

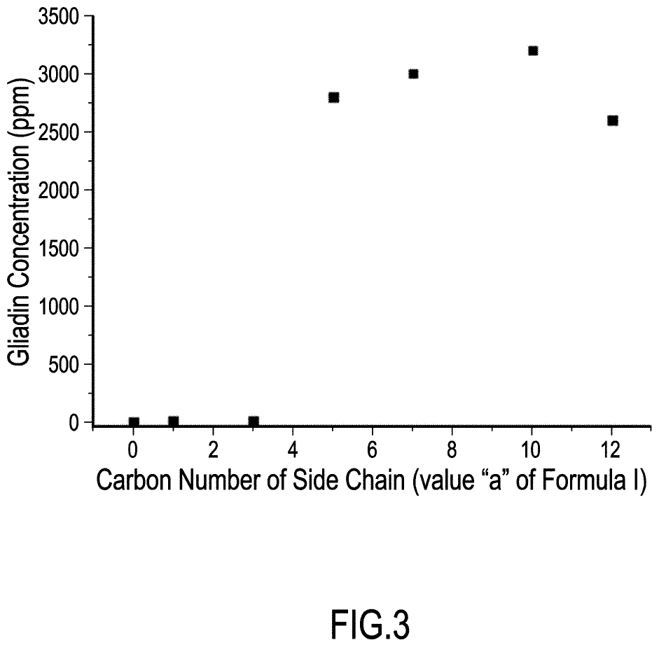

Similar with Test Example 1, except that 3 g of gliadin was added into 10 mL of 1 wt % ionic solution of [DMeIm][OMs](value “a” of Formula I is 0), [TMeIm][OMs] (a=1), [DMePrIm][OMs] (a=3), [DMePIm][OMs] (a=5), [DMeHpIm][OMs] (a=7), [DMeNnIm][OMs] (a=9) or [DMeDdIm][OMs] (a=12). As shown in FIG. 3, gliadin had a higher solubility in the solubility of [DMePIm][OMs] (a=5), [DMeHpIm][OMs] (a=7), [DMeNnIm][OMs] (a=32 9) and [DMeDdIm][OMs] (a=12) ionic solutions, in which the corresponding groups attached on 3-position of the 1,2-dimethyl imidazolium had different carbon numbers (value “a” of Formula I) of 5, 7, 9 and 12, respectively. The [DMeDdIm][OMs] (a=12) ionic solution had a lower solubility but a higher viscosity, which might be disadvantageous.

### Test Example 4: Solubility of Gliadin in 1 wt % Ionic Solutions of Ionic Compounds with Different Anions

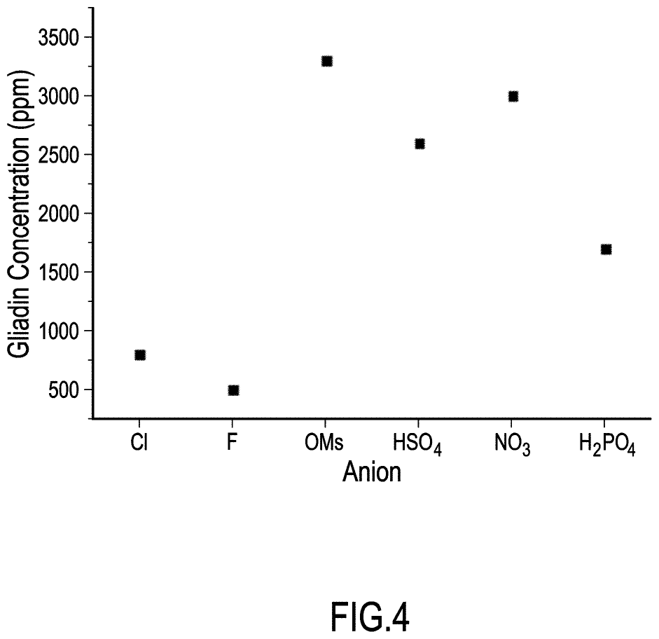

Similar with Test Example 1, except that 3 g of gliadin was respectively added into 10 mL of 1 wt % ionic solutions of [DMePIm][Cl], [DMePIm][F], [DMePIm][OMs], [DMePIm][NO₃], [DMePIm][H₂PO₄] and [DMePIm][HSO₄]. As shown in FIG. 4, the solubility of gliadin in the solutions of ionic compounds with a halogen anion was low, and the ionic compound with a [OMs] anion gave gliadin the greatest solubility.

### Test Example 5: Biochemical Analysis of Gliadin resolved in 1 wt % Ionic Solutions of [DMePIm][OMs]

1 wt % ionic solution of [DMePIm][OMs] in water was prepared first. Specific amounts of gliadin were added into the 1 wt % of ionic solution of [DMePIm][OMs] to prepare gliadin sample solutions with 1 ppm, 2 ppm, 3 ppm, 5 ppm, 7.5 ppm, 10 ppm, 20 ppm and 40 ppm of gliadin. After that, the concentrations of gliadin were confirmed by Wheat/Gluten (Gliadin) ELISA Kit (Crystal chem, AOAC No. 011804).

A 96-well empty ELISA plate was coated with 1 microgram per milliliter (μg/mL) anti-gliadin mouse IgG (2F, purchased from Antaimmu) and then blocked with 1% bovine serum albumin (BSA) to obtain an ELISA plate ready for gliadin examination. The gliadin sample solutions were respectively added into the wells on the ELISA plate to react for 5 min, and the wells were washed by phosphate buffered saline (PBS). After removal of excess solution, 0.1m/mL anti-gliadin human IgA-HRP (anti-gliadin human IgA was 3B7 purchased from Leadgene, and horseradish peroxidase (HRP) was added by MagicLink™ HRP Antibody Conjugation Kit) was added to react for 15 min, the plate was washed, TMB/H₂O₂ reagent (T3854, purchased from TCI) was added to react for 10 min, and the 0.5 moles per liter (mol/L) sulfuric acid aqueous solution was added to terminate the reaction. The absorbance at 450 nm of all wells on the plate was examined by an ELISA reader (TECAN Infinite 200 PRO).

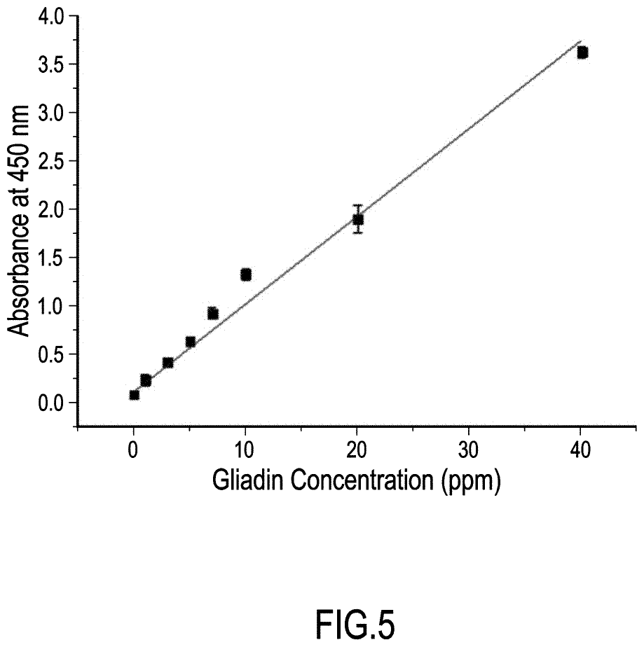

As shown in FIG. 5, the regression line has an R-squared (R²) of 0.9942. This indicates that the gliadin concentration and the absorbance at 450 nm are highly related, and the ELISA plate prepared by the above method can be used for quantification analysis of gliadin.

### Test Example 6: Extraction of Gliadin from Foods by 1 wt % Ionic Solutions of [DMePIm][OMs]

1 wt % ionic solution of [DMePIm] [OMs] in water was prepared first. The hydrophilic proteins were traditionally extracted by an alcohol solution, so a 75 wt % ethanol solution was also prepared for this test.

3 g of bread flour (Blue Jacket Strong Flour, Lien Hwa Milling Corporation) was mixed with 10 mL of 75 wt % ethanol or 10 mL of 1 wt % ionic solution of [DMePIm][OMs] for extraction at room temperature for 5 min to give sample solutions for Test Example 6. The sample solutions were centrifuged at 8500 rpm for 3 min and filtered with a filter with 0.22 μm pore size to obtain filtrate samples. After filtration, Wheat/Gluten (Gliadin) ELISA Kit (Crystal chem, AOAC No. 011804) was used to determine gliadin concentration in the filtrate samples. Similar with Test Example 1, the gliadin concentration was determined by diluted sample solutions with at least 50-fold dilution by the corresponding ionic solution, in order to calculate the gliadin concentration of the sample solutions. In both groups of 75 wt % ethanol and 1 wt % ionic solution of [DMePIm][OMs], the extracted gliadin is higher than 2000 ppm.

In addition, 40 g of dried gluten-free rice noodles (Organic Rice Noodles, YUAN SHUN FOOD CO., LTD.) were soaked in water first to rehydrate the noodles. After rehydration, the rice noodles were drained and soaked in 10 mL of 200 ppm solution of gliadin in 100% ethanol in a container at room temperature to give gliadin-rice noodles. The gluten-free rice noodles had very large specific surface area (total surface area per unit of bulk volume), and most gliadin was adsorbed on the rice noodles. After the solvent ethanol was evaporated, the gliadin-rice noodles were lyophilized in the container to give a gliadin-rice noddle sample. 10 mL of 1 wt % ionic solution of [DMePIm][OMs] (equal volume with the 200 ppm gliadin solution in ethanol) was added to the lyophilized gliadin-rice noodles for extraction at room temperature for 5 min to give sample solutions for Test Example 7. The sample solutions were centrifuged at 8500 rpm for 3 min and filtered with a filter with 0.22 μm pore size to obtain filtrate samples. All groups were repeated for five times. The concentration of gliadin was also determined by Wheat/Gluten (Gliadin) ELISA Kit (Crystal chem, AOAC No. 011804), wherein diluted sample solutions with at least 2-fold dilution by 1 wt % ionic solution of [DMePIm][OMs] were used. The average recovery rates of 75 wt % ethanol and 1 wt % ionic solution of [DMePIm][OMs] were 98.5% and 97.9%, respectively. The recovery rate was calculated by using 200 ppm as 100%.

### Test Example 7: Structure of Gliadin after Extraction

After the gliadin-rice noodle sample extraction of Test Example 6, the extraction solution with about 200 ppm of gliadin was diluted with PBS to 20 μM, as a sample solution. In addition, another sample solution of 20 μM gliadin in PBS was directly prepared by the commercially available gliadin (Leadgene).

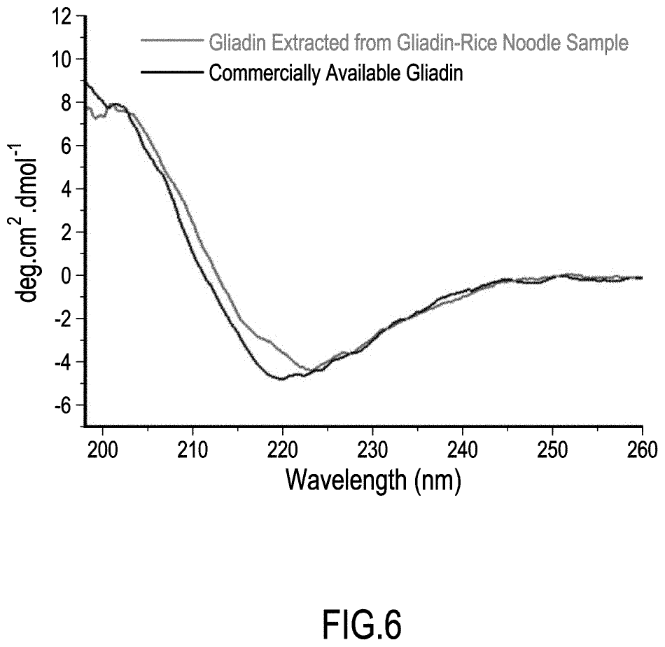

Circular Dichroism Spectrometer (Jasco J-815) was used for structure analysis of the above-mentioned sample solutions obtained by gliadin-rice noodle sample extraction and commercially available gliadin product. As shown in FIG. 6, there was no obvious difference between the extracted gliadin and commercially available gliadin (not extracted by the ion solution of the present invention). In other words, the ionic solution of the present invention did not affect the structure of gliadin.

### Test Example 8: Solubility of Other Hydrophilic Proteins

Many neurodegenerative disorder-related proteins are hydrophobic and can be extracted by alcohol-containing extraction solutions, and the alcohol is disadvantageous for later examinations, especially adverse for the examinations involving antigen-antibody reactions. In this test, two artificially synthesized neurodegenerative disorder-related protein fragments were used for the solubility test:

(1) mutant Huntingtin protein (mHtt):
(KKQQQQQQQQQQQQQQQQQQQQK, SEQ ID No: 1)
(2) beta-amyloid protein (αβ40):
(DAEFRHDSGYEVHHQKLVFFAED
VGSNKGAIIGLMVGGVV, SEQ ID No: 2)

It was known that the recorded solubility of untreated mHtt and αβ40 was less than 0.36 μM in water or PBS, and pre-treatment of hexafluoro-2-propanol (HFIP) could increase their solubility. Similar with Test Example 1, 3 milligrams (mg) of mHtt or αβ40 (excess amount) was added into 10 mL of 1 wt % [DMePIm][OMs] ionic solution, mixed homogenously and stood still for 5 min, centrifuged and filtered to give mHtt and αβ40 sample solutions. In addition, the calibration curves of mHtt and αβ40 were respectively built up from a series of known concentration of HFIP-treated mHtt and αβ40 to the area under curve (AUC) of gel permeation chromatography (GPC). The concentrations of mHtt and αβ40 sample solutions were determined by GPC and their own calibration curves. As the result, the solubility of mHtt and αβ40 in 1 wt % [DMePIm][OMs] ionic solution were both higher than 2 millimolar (mM), which was much higher than the recorded solubility of the untreated mHtt and αβ40.

### Test Example 9: Biocompatibility of Ionic Solutions of [DMePIm][OMs]

Mouse N2a neuroblastoma cells (N2a cells) were cultured and maintained in DMEM High Glucose with L-glutamine and sodium pyruvate (DMEM-HPA-P10, Capricorn Scientific) containing 10% fetal bovine serum (FBS). The N2a cells were seeded in a 6-well plate with a density of 2×10⁵ cells/well overnight. Then the medium was replaced with a testing DMEM High Glucose medium containing 0.1 wt %, 1 wt %, 2 wt %, 5 wt % and 10 wt % of [DMePIm][OMs] and the cells were incubated in an incubator (37° C., 5% CO₂ humidified atmosphere) for 6 h. The testing medium was removed, the cells were washed, and new DMEM High Glucose containing 10% FBS was added and incubated for another 12 h. After that, the cells were subjected to XTT test (X12223, purchased from Thermo Fisher) for determining cell survival rate.

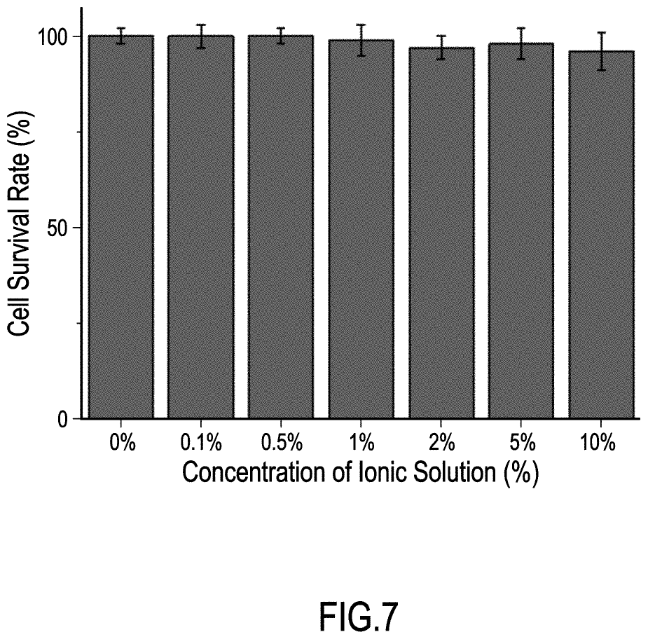

As shown in FIG. 7, the survival rates of cells in the [DMePIm][OMs] ionic solutions at all concentrations were high. This indicates that the ionic compounds of the present invention has good biocompatibility.

### Tests for Zwitterionic Compounds of the Present Invention

### Test Example 10: Coating of Z1 Compound and Coverage Rate

1 mM solution of Z1 Compound in water was prepared and introduced in wells of a 96-well empty ELISA plate (made of polypropylene (PP)) for about 0 min (no coating, used as control), 5 min, 10 min, 25 min, 55 min, 100 min at room temperature for coating, to obtain an ELISA plate coated with the Z1 Compound. After coating, the wells were washed with water, and the coverage rate of Z1 Compound on the surface of the coated wells was determined by atomic force microscopy (AFM) equipped with software of Image J (open-source software developed by National Institutes of Health).

Next, 1 mg/mL of BSA solution was added to the wells to block the deteriorated well surface for 15 min. The BSA solution was then washed by PBS for three times, and then anti-BSA IgG conjugated with HRP (anti-BSA antibody produced in rabbit purchased from Merck, and HRP was added by MagicLink™ HRP Antibody Conjugation Kit) was added to react for 15 min, and the wells were washed again with PBS for three times. After that, TMB/H₂O₂ reagent (T3854, purchased from TCI) was added to react for 10 min, and 0.5 mol/L sulfuric acid aqueous solution was added to terminate the reaction. The absorbance at 450 nm of all wells on the plate was examined by an ELISA reader (TECAN Infinite 200 PRO), and the protein adsorption rate was calculated. The protein adsorption of the plate with no coating (control) was defined as 100%, and protein adsorption rate is calculated accordingly

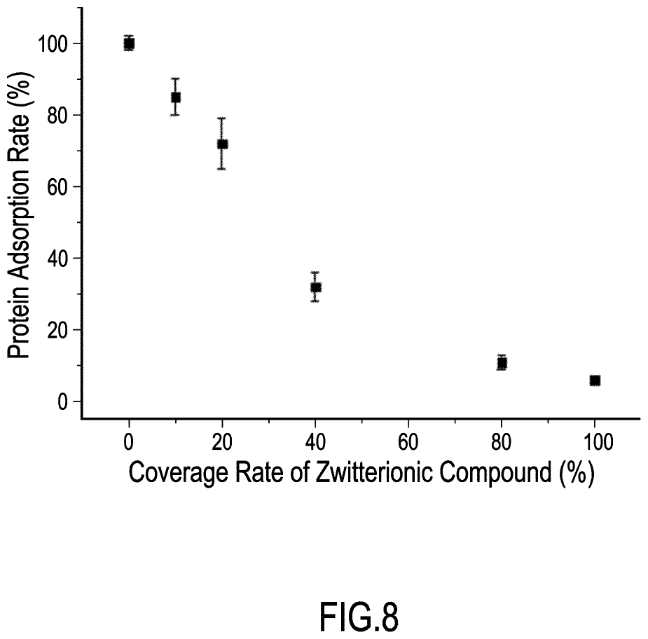

As shown in FIG. 8, higher coverage rate of Z1 Compound resulted in lower protein adsorption rate.

### Test Example 11: Contact Angle Determination of Surface Coated with Z1 Compound

1 mM solution of Z1 Compound in water was prepared first. polystyrene sheets were soaked in the 1 mM solution of Z1 Compound for about 0 min, 5 min, 10 min, 25 min, 55 min, 100 min at room temperature for coating, to obtain polystyrene sheets coated with the Z1 Compound with different coverage rates (confirmed with AFM). After coating, the sheets were washed with water, and the coverage rate was determined by the method disclosed in Test Example 10. An addition, a drop of 20 μL deionized (DI) water was respectively added on the coated surface of the polystyrene sheets, and contact angle was measured by a contact angle meter (DMe-211, Kyowa Interface Science Co. Ltd.).

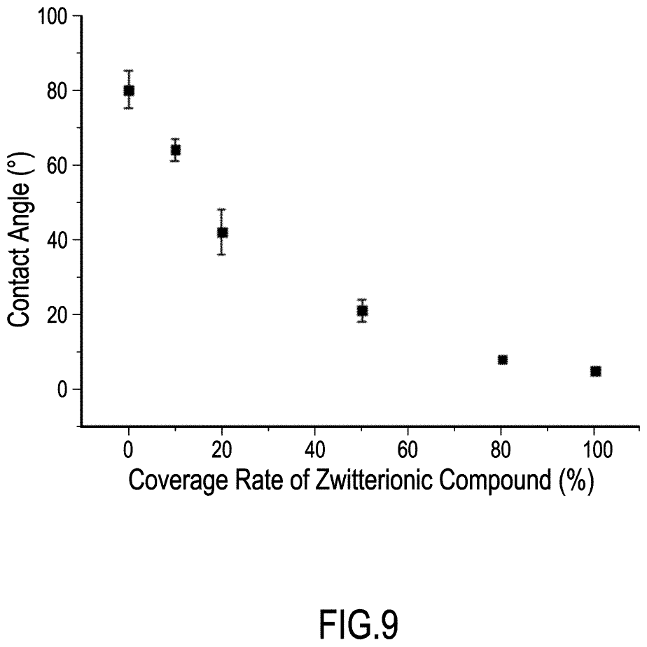

As shown in FIG. 9, the contact angle was lowered when the coverage rate was increased. In addition, when the coverage rate reached 80% or more, the contact angle is less than 10°, which was a hydrophilic characteristic advantageous for examination.

### Test Example 12: Deterioration Test for Surface Coated with Zwitterionic Compound by Using Strong Alkali Solution

An ELISA plate coated with the Q-amine compound with a coverage rate of 100% was prepared by the method disclosed in Test Example 10, except that the Q-amine compound was used to replace the Z1 Compound.

1 molar (M) sodium hydroxide (NaOH) aqueous solution was prepared and the 1 M NaOH solution was added into the ELISA plates coated with the Z1 Compound (prepared in Test Example 10) and the Q-amine compound with an amount of 300 microliters (μL) per well and stood still at room temperature for 5 min, 10 min, 20 min, 30 min, 50 min or 100 min, to deteriorate the coating layer on the surface of the wells. After that, the 1 M NaOH solution was removed and the wells were washed with PBS for three times.

The anti-protein adsorption rate of the ELISA plates coated with the Z1 Compound and the Q-amine compound was calculated by the method disclosed in Test Example 10, except the anti-protein adsorption rate was calculated at last. The protein adsorption of the plate with no coating (control) was defined as 100%, and the anti-protein adsorption rate was defined as 1—protein adsorption rate (in %).

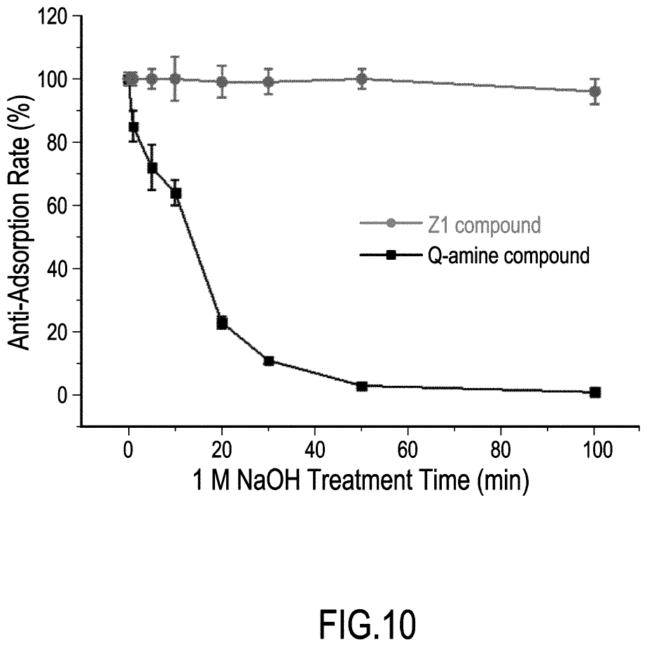

As shown in FIG. 10, the coating layer formed by the Q-amine compound was deteriorated by the treatment of 1 M NaOH solution for 30 min. On the contrary, the coating layer formed by the Z1 compound was not deteriorated even after 100-min 1 M NaOH treatment. Therefore, the Z1 compound is obviously more stable than the Q-amine compound.

### Test Example 13: Deterioration Test for Surface Coated with Zwitterionic Compound by Using Buffers with Different pH Values

Similar to Test Example 12, 1 mM solutions of Z1 Compound and the Q-amine compound were prepared. Glass sheets were soaked in the 1 mM solution of Z1 Compound or the Q-amine compound for 12 h at room temperature for coating, to obtain glass sheets coated by the Z1 Compound or the Q-amine compound with coverage rate of 100%. After coating, the sheets were washed and dried.

1 M NaOH solution was prepared, and one set of the coated glass sheets were soaked in the 1 M NaOH solution at room temperature for 30 min to deteriorate the coating layer on the surface of the glass sheets. After that, the 1 M NaOH solution was removed and the sheets were washed with water for three times and dried. Before and after 1 M NaOH treatment, the contact angles were measured by the method described in Test Example 11, except using PBS with different pH values (pH 2, 4, 6, 8, 10 and 12) to replace the DI water.

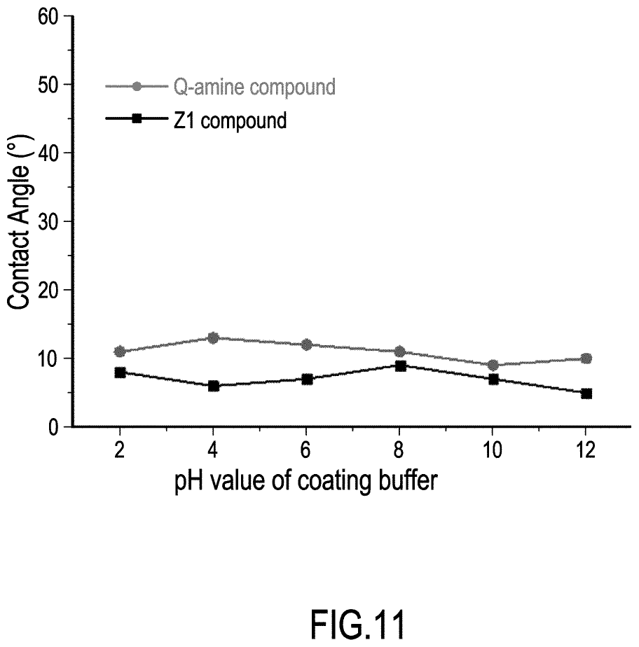

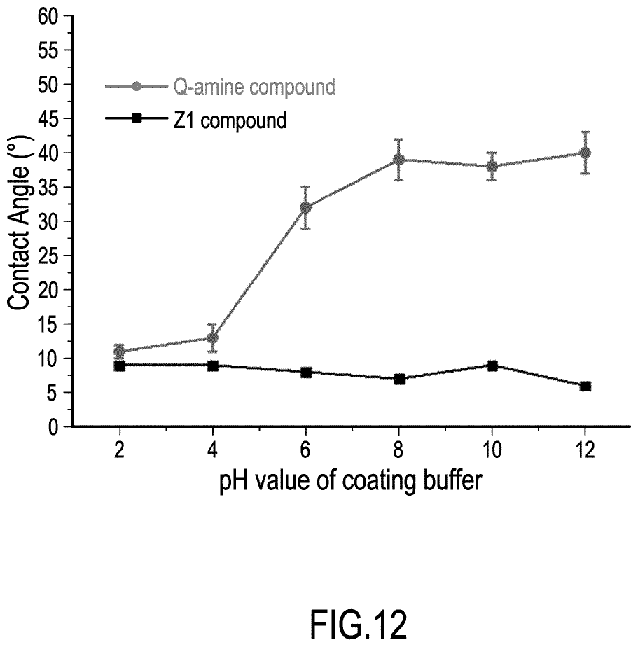

As shown in FIG. 11, the coating layers of both Z1 Compound and the Q-amine compound without 1 M NaOH treatment had a contact angle of about 10° using PBS at all pH values, which was a hydrophilic characteristic. As shown in FIG. 12, however, the coating layer formed by the Q-amine compound was deteriorated by 1 M NaOH treatment and tertiary amine groups were formed on the surface. According to Hofmann elimination reaction, the tertiary amine groups reacted with hydrogen ions in water to form an amine salt under acidic condition, so the amine salts could not be formed when the surface at a higher pH value. Therefore, the contact angle increased when the pH value of the PBS for testing increased. On the contrary, the coating layer formed by the Z1 compound was not deteriorated even after 1 M NaOH treatment. Therefore, the Z1 compound is obviously much more stable than the Q-amine compound.

### Test Example 14: Application of Z1 Compound for Biochemical Analysis

1 mM solution of Z1 Compound in water was prepared first. The 1 mM solution of Z1 Compound was introduced in wells of a 96-well empty ELISA plate for 12 h at room temperature for coating, to obtain an ELISA plate coated by the Z1 Compound with coverage rate of 100%. After coating, the wells were washed with PBS, and then PBS containing 1 mM of 1-ethyl-3-(3-dimethylaminopropyl)-carbodiimide (EDC) (CAS No. 25952-53-8, purchased from Sigma-Aldrich) and 1 mM of N-hydroxysuccinimide (NHS) (CAS No. 6066-82-6, purchased from Merck) was added to activate the —COOH groups on the surface of the coating layer for 15 min. Then the wells were washed with DI water, and 1 μg/mL of anti-mouse IgG (Fc specific Anti-Mouse IgG antibody produced in goat, M2650, Merck) was added to fix the anti-mouse IgG through EDC/NHS on the surface of wells for 5 min. The unbound antibody was washed away by PBS, and 1 μg/mL anti-gliadin mouse IgG conjugated with HRP (anti-gliadin mouse IgG was 2F purchased from Antaimmu, and HRP was added by MagicLink™ HRP Antibody Conjugation Kit) was added to react for 10 min. The plate was washed, TMB/H₂O₂ reagent (T3854, purchased from TCI) was added to react for 10 min, and 0.5 mol/L sulfuric acid aqueous solution was added to terminate the reaction. The absorbance at 450 nm of all wells on the plate was examined by an ELISA reader (TECAN Infinite 200 PRO).

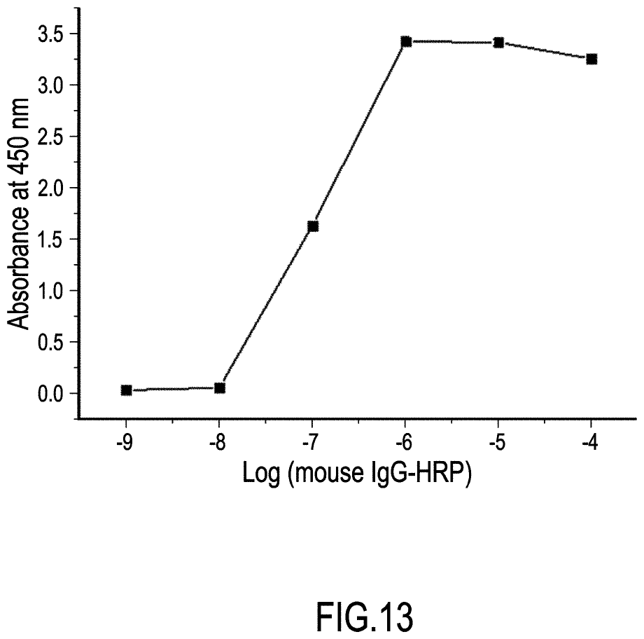

As shown in FIG. 13, in which the X axis showed the log value of the absorbance at 450 nm, it was obviously that the coating layer formed by the Z1 compound not only had good anti-nonspecific protein adsorption effect, but also could be applied to biochemical analysis using antibodies for examination.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

element.

What is claimed is:

1. An ionic compound represented by the following Formula I: 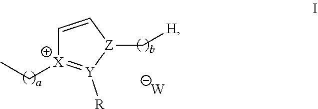
wherein,
X is N;
Y is C or N;
Z is N;
R is H, —CH₃, —C₂H₅, —C₃H₇ or —C₄H₉;
a is an integer of 1 to 40;
b is an integer of 0 to 40; and
W⁻ is selected from the group consisting of F⁻, Cl⁻, OMs⁻, NTf₂⁻, HSO₃⁻, HSO₄⁻, NO₃⁻, NO₂⁻ and H₂PO₄⁻.

2. The ionic compound according to claim 1, wherein Y is C; X is N; and Z is N.

3. The ionic compound according to claim 2, wherein a is an integer of 3 to 15; and b is an integer of 0 to 5.

4. An ionic solution for extracting a hydrophobic protein, comprising the ionic compound according to claim 1 and a solvent.

5. The ionic solution according to claim 4, wherein Y is C; X is N; and Z is N.

6. The ionic solution according to claim 5, wherein a is an integer of 3 to 15; and b is an integer of 0 to 5.

7. The ionic solution according to claim 4, wherein the solvent is water, and the ionic compound is in an amount of 0.1% by weight to 10% by weight.

8. The ionic solution according to claim 5, wherein the solvent is water, and the ionic compound is in an amount of 0.5% by weight to 2% by weight.

9. The ionic solution according to claim 4, wherein the hydrophobic protein is gliadin, mutant Huntingtin protein, or beta-amyloid protein.

10. An anti-nonspecific protein adsorption zwitterionic compound represented by the following Formula II: 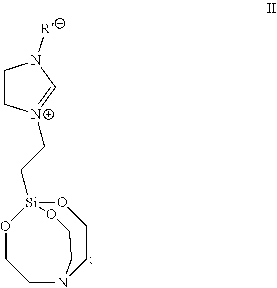
wherein,
R′ is 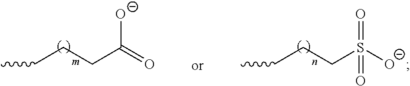
m is an integer of 0 to 7; and
n is an integer of 1 to 7.

11. The zwitterionic compound according to claim 10, wherein R′ is 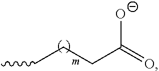
and m is an integer of 0 to 3.

12. The zwitterionic compound according to claim 10, wherein R′ is 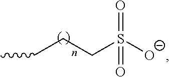
and n is an integer of 1 to 3.

13. An anti-nonspecific protein adsorption element comprising a substrate and an anti-nonspecific protein adsorption layer, wherein the substrate is coated with the zwitterionic compound according to claim 10 to form the anti-nonspecific protein adsorption layer.

14. The element according to claim 13, wherein the substrate is made of plastic, metal or metal oxide.

15. The element according to claim 13, wherein the anti-nonspecific protein adsorption layer has a coverage rate of 80% to 100% on the substrate.

16. The element according to claim 13, wherein the anti-nonspecific protein adsorption layer has a contact angle of 10° or less.
